(19) United States
(12) Patent Application Publication
(10) Pub. No.: US 2012/0172674 A1
(43) Pub. Date: Jul. 5, 2012

(54) SYSTEMS AND METHODS FOR CLINICAL DECISION SUPPORT

(75) Inventors: Andreas Welz, Dorndstadt (DE); Arno Heikela, Helsinki (FI)

(73) Assignee: General Electronic Company, Schenectady, NY (US)

(21) Appl. No.: 12/982,379

(22) Filed: Dec. 30, 2010

(51) Int. Cl. A61B 5/00

(52) U.S. Cl. 600/300

(57) ABSTRACT

Systems and methods for providing clinical decision support are provided. It is determined whether a health attribute qualifies for a condition for which a care provider is to be notified. When the health attributes qualifies for such a condition, a patient indication is provided. A first care protocol that includes a plurality of rules and that corresponds to the patient indication is selected. From the plurality of rules, a first plurality of treatment-observation options is generated. Each of the first plurality of treatment-observation options is displayed. A first user-generated response that corresponds to at least one of the plurality of treatment-observation options is received. A treatment recommendation is determined based at least in part on the first user-generated response. The treatment recommendation is displayed. Feedback is accepted with respect to the treatment recommendation.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND

Hospitals and clinicians today are facing pressure to deliver high quality patient care, prevent adverse events/errors, and implement clinical best practices while reducing the cost of healthcare delivery. Furthermore, hospitals can face dramatic variation in clinical demand and are increasingly likely to be declined reimbursement when patient care falls short. Hospitals that operate at or over capacity may experience heightened rates of safety events. Current support is provided based on anticipated, static events, and do not account for chaos and unpredictability associated with many medical events.

### BRIEF SUMMARY

Certain examples of the present invention provide systems and methods for providing clinical decision support.

Certain examples provide a computer-implemented method for providing clinical decision support. The method includes receiving a plurality of care protocols and at least one health attribute of a patient. The method also includes determining whether the at least one health attribute qualifies for a condition for which a care provider is to be notified. The method further includes providing via a user interface a patient indication, when the at least one health attribute qualifies for the condition, to notify the care provider that the patient qualifies for a care treatment. The method also includes selecting from the plurality of care protocols a first protocol that corresponds to the patient indication and that includes a first plurality of rules. The method further includes generating from the first plurality of rules a first plurality of treatment-observation options. The method also includes displaying via the user interface each of the first plurality of treatment-observation options and a suggestion regarding a recommended treatment-observation option, to enable the care provider to determine which of the first plurality of treatment-observation options applies to the patient. The method further includes receiving a first user-generated response that corresponds to at least one of the first plurality of treatment-observation options. The method also includes determining a treatment recommendation based at least in part on the first user-generated response. The method further includes displaying via the user interface the treatment recommendation to notify the care provider of the treatment recommendation for the care treatment. The method also includes accepting feedback with respect to the treatment recommendation applied to the patient.

Certain examples provide a clinical decision support system. The system includes a processor connected to a memory. The processor is programmed to implement the system. The system also includes a database interface to receive a plurality of care protocols and at least one health attribute of a patient. The system further includes a decision module. The decision module is to determine whether the at least one health attribute qualifies for a condition for which a care provider is to be notified. The decision module is to select from the plurality of care protocols a first protocol that corresponds to the patient indication and that includes a first plurality of rules. The decision module is to generate from the first plurality of rules a first plurality of treatment-observation options. The decision module is to determine a treatment recommendation based at least in part on a first user-generated response that corresponds to at least one of the first plurality of treatment-observation options. The system also includes a user interface. The user interface is to provide the patient indication when the at least one health attribute qualifies for the condition to notify the care provider that the patient qualifies for a care treatment. The user interface is to display each of the first plurality of treatment-observation options. The user interface is to enable the care provider to determine which of the first plurality of treatment-observation options applies to the patient. The user interface is to receive the first user-generated response. The user interface is to display the treatment recommendation to notify the care provider of the treatment recommendation for the care treatment. The user interface is to accept feedback with respect to the treatment recommendation applied to the patient.

Certain examples provide a tangible computer-readable storage medium. The storage medium includes a set of instructions that, when executed, cause a processor to at least receive a plurality of care protocols and at least one health attribute of a patient. The processor also determines whether the at least one health attribute qualifies for a condition for which a care provider is to be notified. The processor further provides via a user interface a patient indication, when the at least one health attribute qualifies for the condition, to notify the care provider that the patient qualifies for a care treatment. The processor also selects from the plurality of care protocols a first protocol that corresponds to the patient indication and that includes a first plurality of rules. The processor also generates from the first plurality of rules a first plurality of treatment-observation options. The processor further displays via the user interface each of the first plurality of treatment-observation options, to enable the care provider to determine which of the first plurality of treatment-observation options applies to the patient. The processor also receives a first user-generated response that corresponds to at least one of the first plurality of treatment-observation options. The processor further determines a treatment recommendation based at least in part on the first user-generated response. The processor also displays via the user interface the treatment recommendation to notify the care provider of the treatment recommendation for the care treatment. The processor further accepts feedback with respect to the treatment recommendation applied to the patient.

### BRIEF DESCRIPTION OF SEVERAL VIEWS OF THE DRAWINGS

FIG. 1A illustrates an environment in which an example clinical decision support system operates.

FIG. 1B illustrates an example classification for kidney failure.

FIG. 1C illustrates a portion of an example care protocol that corresponds to the example classification for kidney failure.

FIG. 2 illustrates an example log-in interface of the example clinical decision support system.

FIG. 3 illustrates an example department interface of the example clinical decision support system.

FIG. 4 illustrates an example patient interface of the example clinical decision support system.

FIG. 5 illustrates the example patient interface after a care provider selects a first treatment-observation option.

FIG. 6 illustrates the example patient interface after the care provider selects a second treatment-observation option.

FIG. 7 illustrates the example patient interface showing a graphical representation of a care protocol.

FIG. 8 illustrates the example patient interface showing an example protocol table.

FIG. 9 illustrates a flow diagram depicting an example method for clinical decision support.

FIG. 10 illustrates a block diagram of an example processor system that can be used to implement the apparatus and methods described herein.

The foregoing summary, as well as the following detailed description of certain examples of the present invention, will be better understood when read in conjunction with the appended drawings. For the purpose of illustrating the invention, certain examples are shown in the drawings. It should be understood, however, that the present invention is not limited to the arrangements and instrumentality shown in the attached drawings.

### DETAILED DESCRIPTION OF CERTAIN EMBODIMENTS

Although the following discloses example methods, systems, articles of manufacture, and apparatus including, among other components, software executed on hardware, it should be noted that such methods and apparatus are merely illustrative and should not be considered as limiting. For example, it is contemplated that any or all of these hardware and software components could be embodied exclusively in hardware, exclusively in software, exclusively in firmware, or in any combination of hardware, software, and/or firmware. Accordingly, while the following describes example methods, systems, articles of manufacture, and apparatus, the examples provided are not the only way to implement such methods, systems, articles of manufacture, and apparatus.

When any of the appended claims are read to cover a purely software and/or firmware implementation, at least one of the elements in an at least one example is hereby expressly defined to include a tangible medium such as a memory, DVD, CD, etc. storing the software and/or firmware.

Hospitals and clinicians today face pressure to deliver high quality patient care, prevent adverse events/errors, and implement clinical best practices while reducing the cost of healthcare delivery. Furthermore, hospitals can face dramatic variation in clinical demand and increasingly face a likelihood of being turned down for reimbursement when patient care falls short. Hospitals that operate at or over capacity may experience heightened rates of safety events and might consider re-engineering the structures of care to respond better during periods of high stress.

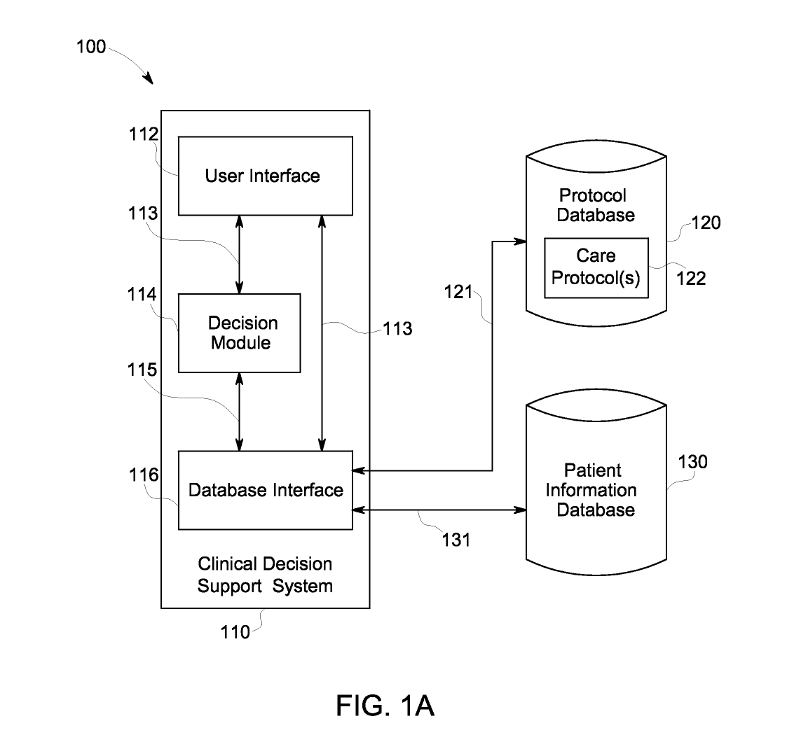

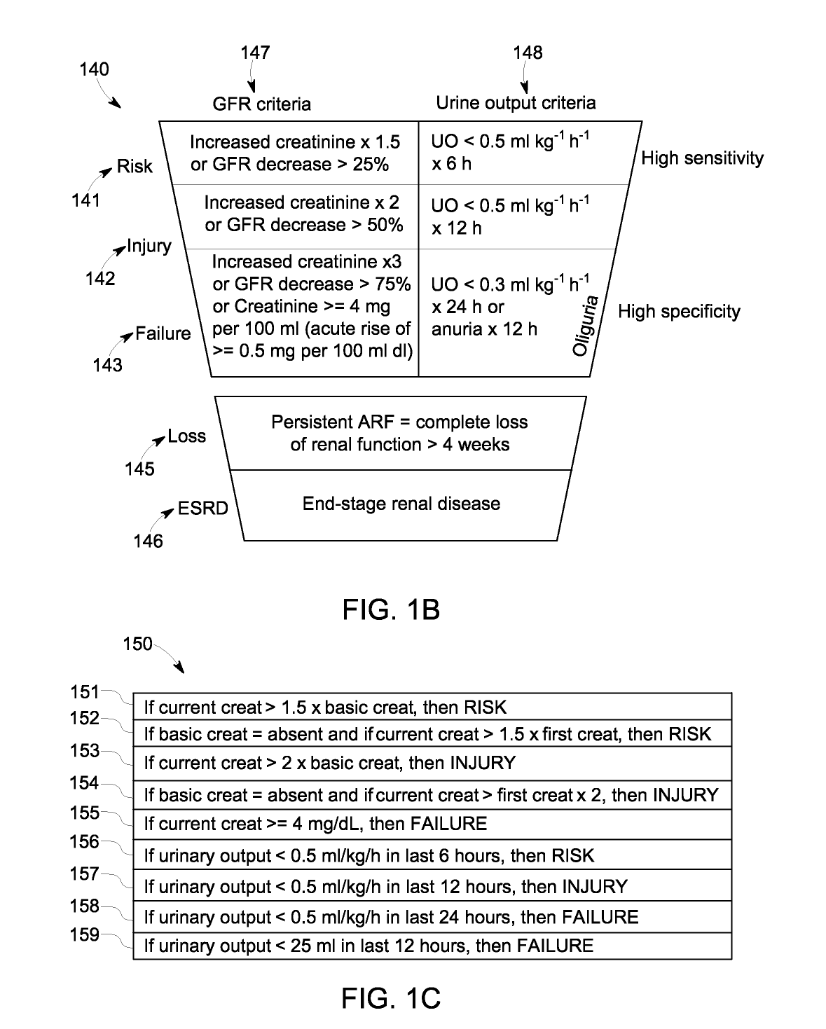

FIG. 1A shows an example processor-implemented environment 100. The environment 100 includes a clinical decision support system 110, a protocol database 120, and a patient information database 130. The clinical decision support system 110 includes a user interface 112, a decision module 114, and a database interface 116. The user interface 112 is logically connected to the decision module 114 and to the database interface 116, as indicated by arrows 113. The decision module 114 is logically connected to the database interface 116, as indicated by arrow 115.

The protocol database 120 stores one or more care protocols 122. A care protocol is generally a detailed guideline for how to manage (e.g., identify and/or treat) a clinical problem, and/or a patient condition. The one or more care protocols 122 generally includes a plurality of rules. The format of the one or more care protocols 122 can vary from plain text to an algorithmic sequence of decisions.

The patient information database 130 stores patient-specific attributes. The patient-specific attribute includes at least one attribute specific to a patient. Suitable patient attributes include, but are not limited to, blood pressure, body temperature, and/or heart rate. The patient information database 130 can store various other patient attributes.

In an example, the one or more care protocols 122 are created by responsible care giver(s) based on statistical data derived from a population of patients that does not include the specific patient. The population of patients may include patients whose clinical problem corresponds to the specific patient's clinical problem, or the population may include patients having various clinical problems. Statistical data and one or more rules can be used to generate a care protocol, for example.

The database interface 116 of the clinical decision support system 110 is configured to receive information from, and send information to, the protocol database 120, as indicated by arrow 121. For instance, the database interface 116 can receive one or more of the care protocols 122 from, or send one or more care protocols to, the protocol database 120. The database interface 116 is further configured to receive information from, and send information to, the patient information database 130, as indicated by arrow 131. For instance, the database interface 116 can receive patient information from, and/or send patient information to, the patient information database 130.

FIGS. 1B and 1C together show a medical model and its representation as a care protocol, which can be stored to the protocol database 120. FIG. 1B shows a Risk, Injury, Failure, Loss, and End-Stage Kidney (RIFLE) diagram 140. The RIFLE diagram 140 classifies acute kidney injury according to three grades of increasing severity: risk 141, injury 142, and failure 143. The RIFLE diagram 140 also classifies acute kidney injury according to two outcome classes: loss 145 and end-stage kidney disease 146. The grades of severity 141-143 are based on one of two criteria: Glomerular filtration rate (GFR) 147 (the flow rate of filtered fluid through the kidney) or urine output 148. The RIFLE diagram 140 presents one or more guidelines at the intersection of each grade of severity 141-143 and each criterion 147-148. For instance, at the intersection of risk 141 and GFR 147, the RIFLE diagram 140 provides the guideline “Increased creatinine×1.5 or GFR decrease>25%.”

FIG. 1C shows a portion of an example care protocol 150 that corresponds to the RIFLE diagram 150. The partial care protocol 150 is an example of a care protocol that can be stored to the protocol database 120. The partial protocol 150 includes several rules 151-159. For instance, rule 151 corresponds to the part of the guideline in the RIFLE diagram 150 at the intersection of risk 141 and GFR 147. Rule 151 provides that if a patient's current creatinine level exceeds 150% of the basic creatinine level, then the patient will be classified in the risk category. The basic creatinine level is a level to be determined by a physician. Rules 152-159 derive from corresponding guidelines in the RIFLE diagram 140.

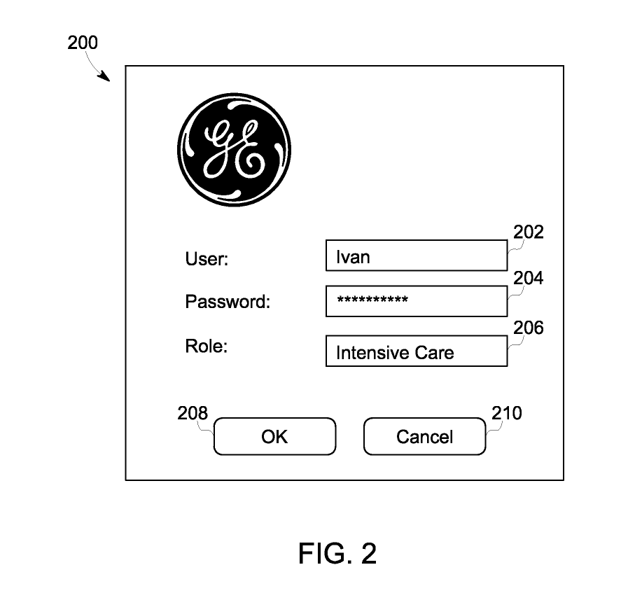

In an example, the user interface is a graphical software module. FIGS. 2-8 illustrate screenshots of the example user interface 112 of the example clinical decision support system 110. The user interface may be implemented in various other ways. For instance, the user interface can be a command-prompt-driven interface.

FIG. 2 shows a log-in screen 200, which serves as a gateway to the user interface 112. The log-in screen 200 includes a user field 202, a password field 204, a role field 206, an OK button 208, and a CANCEL button 210. The user field 202 indicates a user's name, the password field 204 indicates the user's security credential, and the role field 206 indicates the user's organizational role. A user can submit his name into the user field 202, his password into the password field 204, and his organizational role into the role field 206. Then, the user can select the OK button 208. If the user's credentials are recognized, the user interface 112 proceeds; otherwise, the user interface 112 may prompt the user to re-submit his or her credentials.

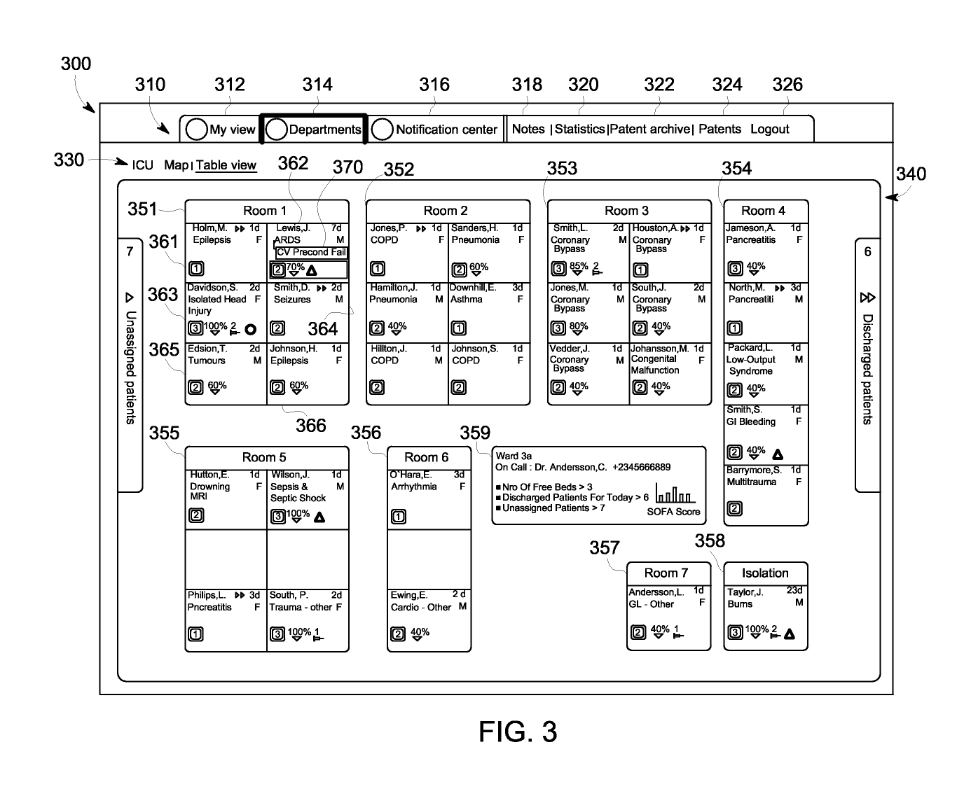

FIG. 3 shows a department screen 300 of the user interface 112. The department screen 300 includes a top panel 310. The top panel 310 has a My View tab 312, a Departments tab 314, a Notification Center tab 316, a Notes tab 318, a Statistics tab 320, a Patient Archive tab 322, a Patients tab 324, and a Logout tab 326. Below the top panel 310 is a view option 330, which enables a user to display the department screen 300 in a map view or a table view. The shown department screen 300 is displayed in map view. Below the view option 330 is a main body portion 340. The main body portion 340 generally depicts various rooms, patients occupying those rooms, indicators relating to those patients, and other information. In particular, the main body portion 340 shows nine room icons 351-359. Room icon 359 represents an example ward that has at least three free beds, at least six discharged patients on a given day, and at least seven unassigned patients on that day. Each of room icons 351-358 represents an example room with at least one patient. For instance, room icon 351 has six patient icons 361-366; therefore, room icon 351 represents a room with six patients. The patient in the room corresponding to room icon 362, for example, is a male named “Lewis, J.,” who has been admitted for seven days for acute respiratory distress syndrome (abbreviated in FIG. 2 as “ARDS”).

In addition to this information, the patient icon 362 shows a patient indication 370, titled “CV Precond fail.” The patient indication 370 generally appears in response to a patient-related event. In this case, the patient indication 370 appears in the patient icon 362 to indicate that “Lewis, J.” has experienced a cardiovascular failure (abbreviated in the patient indication 370 as “CV Precond fail”).

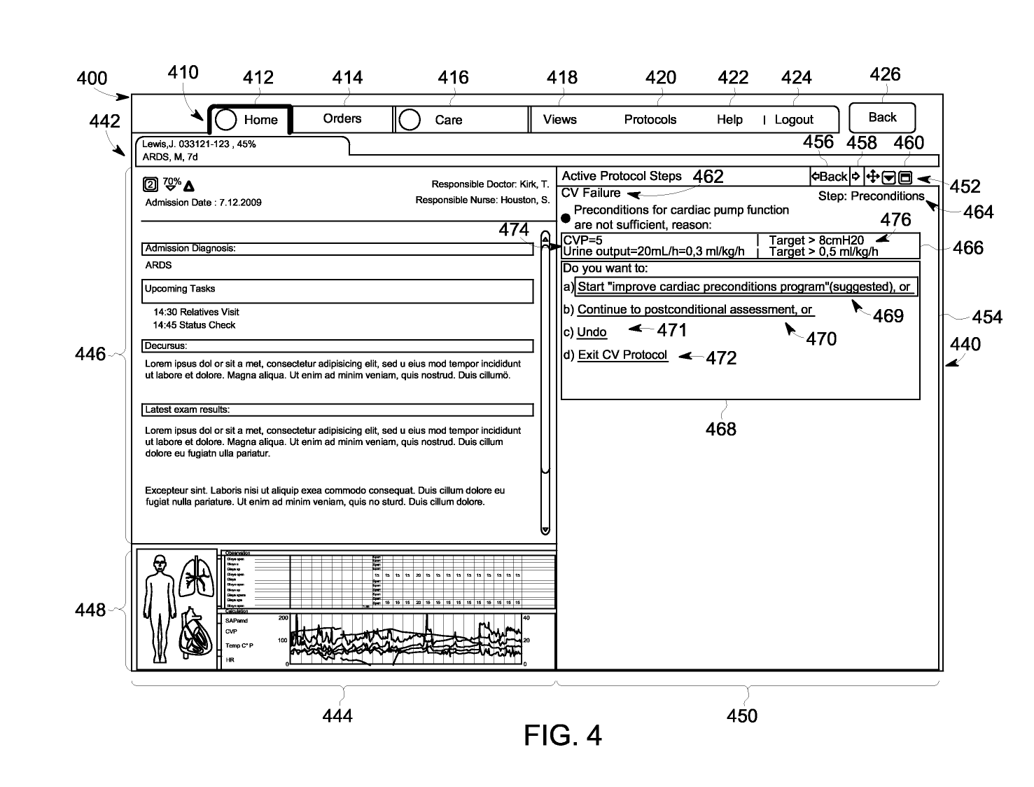

FIG. 4 shows a patient screen 400 of the user interface 112. The shown patient screen 400 is accessible by selecting patient icon 362 in the main body portion 340 of the department screen 300. The patient screen 400 includes a top panel 410. In the top panel 410 are seven tabs, a Home tab 412, an Orders tab 414, a Care tab 416, a Views tab 418, a Protocols tab 420, a Help tab 422, and a Logout tab 424. To the right of the top panel 410 is a Back button 426.

Below the top panel 410 is a main body portion 440 of the patient screen 400. The main body portion 440 has a tab 442 that identifies the patient, in this case “Lewis, J.,” and includes other patient-related information. Though not shown, the patient screen 400 may include multiple main body portions, each accessible via a corresponding one of multiple tabs. The main body portion 440 is vertically split into a patient information portion 444 and a patient protocol portion 450.

The patient information portion 444 is split horizontally into a general information sub-portion 446 and a vitals sub-portion 448. The general information sub-portion 446 generally includes attributes specific to the patient. These attributes may be stored to the patient information database 130, and the user interface 112 may be configured to obtain the attributes from the patient information database 130. As shown, the general information sub-portion 446 lists various attributes, including, among others, the patient's admission date, his responsible doctor and nurse, his admission diagnosis, and his latest exam results. Of course, the patient information sub-portion 446 may list other suitable patient attributes.

The vitals sub-portion 448 lists and graphically depicts relevant patient vitals attributes. Like the attributes that form the general information sub-portion 446, the attributes that form the vitals sub-portion 448 may be stored to the patient information database 130, which is accessible to the user interface 112 via the database interface 116. As shown, the vitals sub-portion 448 depicts and lists various attributes, including the patient's body temperature, his heart rate, and the like. The vitals sub-portion 448 may, for example, list or depict other suitable patient attributes.

The patient protocol portion 450 includes a toolbar sub-portion 452 and a body sub-portion 454. The toolbar sub-portion 452 includes, among other buttons, a back button 456, a forward button 458, and a protocol display button 460. The body sub-portion 454 includes information relevant to an active protocol. The shown body sub-portion 454 lists a protocol type 462, an active protocol rule 464, a detailed information dialog 466 relating to the active protocol rule 464, and a treatment decision dialog 468.

A user can select the protocol display button 460 to switch the main body portion 440 of the patient screen 400 from the split mode shown in FIG. 4 to a protocol display mode.

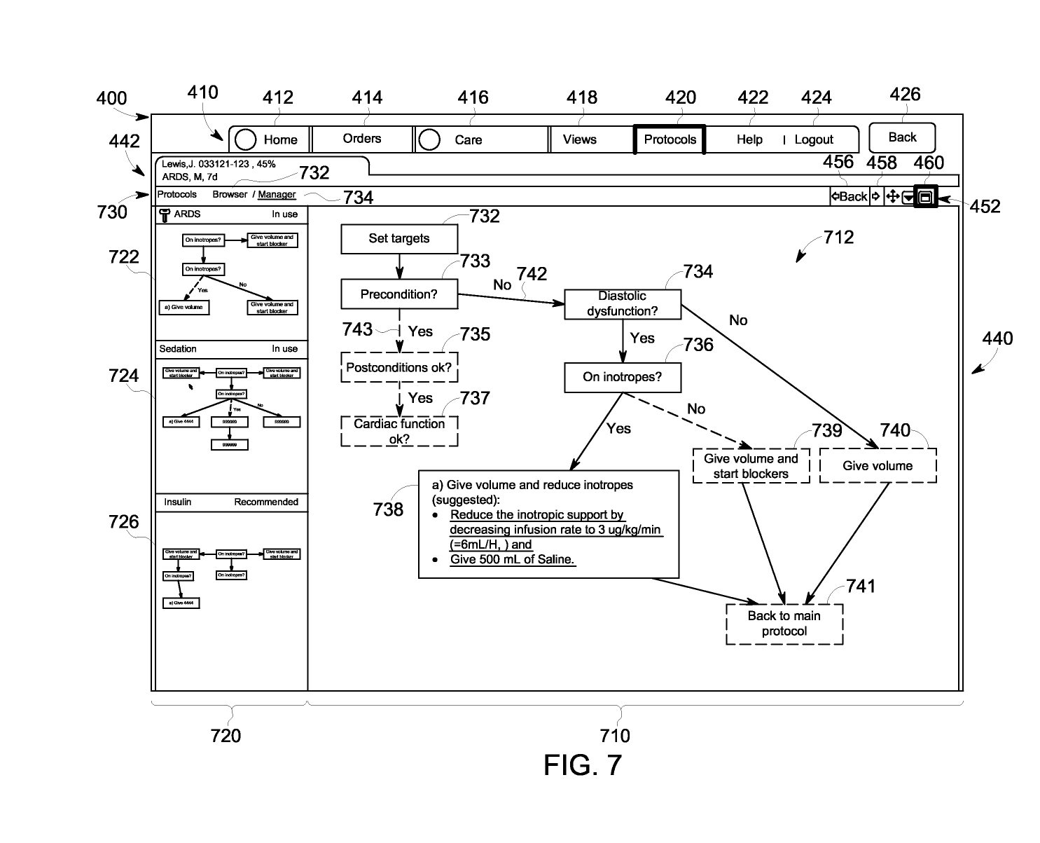

FIG. 7 shows the main body portion 440 of the patient screen 400 in the protocol display mode. In this mode, the main body portion 440 includes an active protocol sub-portion 710 and a subsidiary protocol sub-portion 720. The active protocol sub-portion 710 graphically depicts a flow diagram 712 of the active protocol. The subsidiary protocol sub-portion 720 generally depicts one or more flow diagrams of subsidiary protocols. The shown subsidiary protocol sub-portion 720 graphically depicts a first flow diagram 722, a second flow diagram 724, and a third flow diagram 726 corresponding, respectively, to first, second, and third subsidiary protocols.

The main body portion 440 in protocol display mode also includes a view selection panel 730, which includes a browser link 732 and a manager link 734. When the user selects the browser link 732, the main body portion 440 appears in the protocol display mode, as shown in FIG. 7. When the user selects the manager link 734, the main body portion 440 appears in a protocol manager mode.

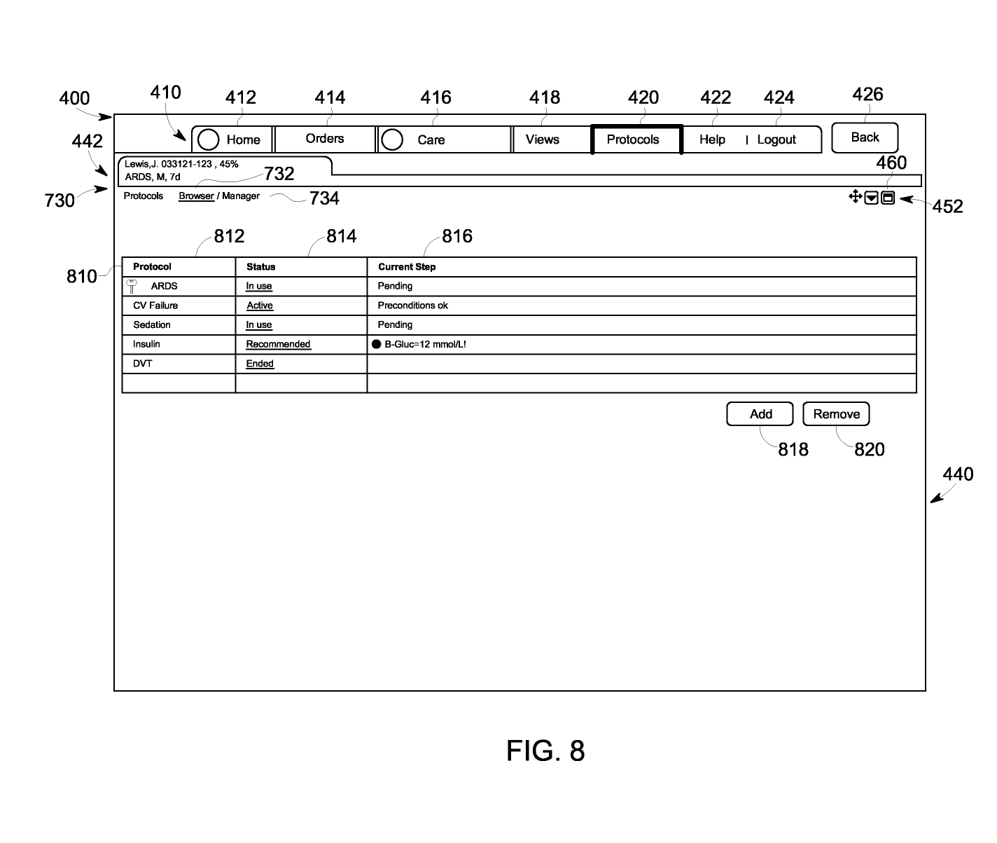

FIG. 8 shows the main body portion 440 of the patient screen 400 in the protocol manager mode. In this mode, the main body portion 440 includes a protocol table 810. The protocol table 810 includes a protocol column 812, a status column 814, and a current rule column 816. The shown example protocol table 810 lists one active protocol (CV failure), two in-use protocols (ARDS and Sedation), one recommended protocol (Insulin), and one ended protocol (DVT). The protocol table 810 can list fewer or more protocols. The main body portion 440 of the patient screen 400 also includes an add button 818, which enables the user to add a protocol to the protocol table 810, and a remove button 820, which enables the user to remove a protocol from the protocol table 810.

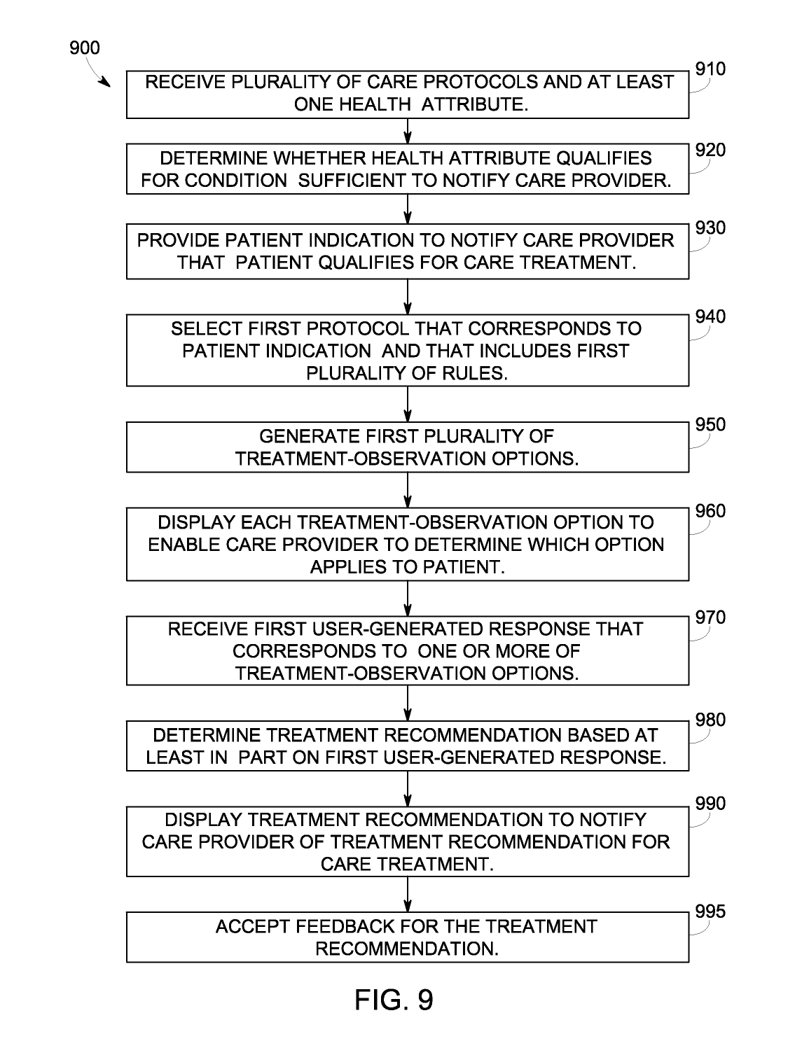

FIG. 9 is a flow diagram representative of example machine readable instructions that can be executed to implement the example systems shown in FIGS. 1-8 and/or portions of one or more of those systems. The example process(es) of FIG. 9 can be performed using a processor, a controller and/or any other suitable processing device. For example, the example process(es) of FIG. 9 can be implemented using coded instructions (e.g., computer readable instructions) stored on a tangible computer readable medium such as a flash memory, a read-only memory (ROM), and/or a random-access memory (RAM). As used herein, the term tangible computer readable medium is expressly defined to include any type of computer readable storage and to exclude propagating signals. Additionally or alternatively, the example process(es) of FIG. 9 can be implemented using coded instructions (e.g., computer readable instructions) stored on a non-transitory computer readable medium such as a flash memory, a read-only memory (ROM), a random-access memory (RAM), a cache, or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

Alternatively, some or all of the example process(es) of FIG. 9 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example process(es) of FIG. 9 can be implemented manually or as any combination(s) of any of the foregoing techniques, for example, any combination of firmware, software, discrete logic and/or hardware. Further, although the example process(es) of FIG. 9 is described with reference to the flow diagram of FIG. 9, other methods of implementing the process(es) of FIG. 9 can be employed. For example, the order of execution of the blocks can be chnged, and/or some of the blocks described can be changed, eliminated, sub-divided, or combined. Additionally, any or all of the example process(es) of FIG. 9 can be performed sequentially and/or in parallel by, for example, separate processing threads, processors, devices, discrete logic, circuits, etc.

FIG. 9 is a flow diagram 900 that shows an example method to provide clinical decision support. The method will be explained with reference to the example clinical decision support system 110 depicted in FIGS. 1-8. However, the method is not limited to the example system 110.

Block 910 generally includes receiving a plurality of care protocols and at least one health attribute of a patient. Refer to the example clinical decision support system 110 shown in FIGS. 1A and 3. As described above, FIG. 3 shows that the main body portion 340 of the department screen 300 includes several room icons 351-359 and at least one patient icon in each of those room icons. For instance, room icon 351 includes six patient icons 361-366. In block 910, the database interface 116 receives from the patient information database 130 patient attributes for the patients corresponding to patient icons 361-366 and for patients in the other rooms 352-359. Suitable patient attributes include, but are not limited to, blood pressure, body temperature, and heart rate. The database interface 116 can receive various other patient attributes. In block 910, the database interface 116 also receives one or more of the care protocols 122 from the protocol database 120. The database interface 116 then passes the patient attributes and the one or more care protocols to the decision module 114.

Referring to FIG. 9, block 920 generally includes determining whether the at least one patient attribute qualifies for a condition for which a care provider is to be notified. Refer to FIGS. 1A and 3. The decision module 114 determines whether the received patient attributes meet a condition for which the care provider is to be notified. In an example, the condition may be a threshold. For instance, if the patient attribute were urine output, then the condition could be a threshold urine output. In another example, the one or more received care protocols may indicate the patient condition.

Referring to FIG. 9, block 930 generally includes providing via a user interface a patient indication, when the at least one health attribute qualifies for the condition, to notify the care provider that the patient qualifies for a care treatment (refer, for example, to FIGS. 1A and 3). Once the decision module 114 determines that the received patient attributes meet a condition for which the care provider is to be notified, the user interface 112 provides a patient indication to notify the care provider that a given patient qualifies for a care treatment. For instance, the decision module 114 has determined that the patient attributes of patient 362, “Lewis, J.,” meet a condition for which the patient's care provider is to be notified. In response, the user interface 112 displays the patient indication 370 to notify Lewis, J.'s care provider that Lewis, J. has experienced a cardiovascular failure (abbreviated as “CV Precond fail”).

The user interface 112 may notify the patient's care provider of the patient indication 370 in various other ways. For example, the user interface 112 may provide the patient indication 370 to the care provider over a network. In particular, the user interface 112 may send the patient indication 370 to the care provider using any combination of an e-mail, an SMS message, and a page, such that the care provider may receive the patient indication 370 at a dedicated local terminal or via a remote or handheld device. These examples are not limiting. The user interface 112 may notify the patient's care provider in various other ways, and the care provider may view the patient indication 370 in various other ways.

Referring to FIGS. 1A, 3, and 9, block 940 generally includes selecting from the plurality of care protocols a first protocol that corresponds to the patient indication and that includes a first plurality of rules. In the example clinical decision support system 110, the decision module 114 has selected from the received care protocols a “CV Failure” protocol that corresponds to the patient indication 370. The CV Failure protocol includes a plurality of rules (to be discussed).

Block 950 generally includes generating from the first plurality of rules a first plurality of treatment-observation options. In the example clinical decision support system 110, the decision module 114 has generated from the CV Failure protocol's rules a plurality of treatment-observation options (to be discussed).

Block 960 generally includes displaying via the user interface each of the first plurality of treatment-observation options to enable the care provider to determine which of the first plurality of treatment-observation options applies to the patient (refer, for example, to FIGS. 1A and 4). The user interface 112 displays the active care protocol (“CV Failure”) 462, the CV Failure protocol's active rule 464 (“Preconditions”), and a treatment decision dialog 468 that includes several treatment-observation options 469-472. The user interface 112 also displays in the detailed information dialog 466 two patient attributes 474 and two target patient attributes 476. Both the shown patient attributes 474 and the shown target patient attributes 476 relate to cardiovascular pressure and urine output. In particular, Lewis, J.'s actual cardiovascular pressure is 5 cmH₂O and his target cardiovascular pressure is 8 cmH₂O; likewise, his actual urine output is 0.3 mL/kg/h and his target urine output is 0.5 mL/kg/h. The display of the patient attributes 474 and the target patient attributes 476 may assist the care provider by notifying him or her that the desirable treatment should attain the values of the target patient attributes.

Referring to FIG. 9, block 970 generally includes receiving a first user-generated response that corresponds to at least one of the first plurality of treatment-observation options (refer, for example, to FIGS. 1A and 4). The care provider may observe the patient and, on that basis, select one of the treatment-observation options 469-472. For instance, the care provider may select option 469 to start a program for improving cardiac preconditions, option 470 to continue to a postconditional assessment, option 471 to undo his or her previous selection, if any, or option 472 to exit the CV Failure protocol. In FIG. 4, option 469 is highlighted to indicate that the care provider selected option 469. The user interface 112 receives the care provider's selection of option 469.

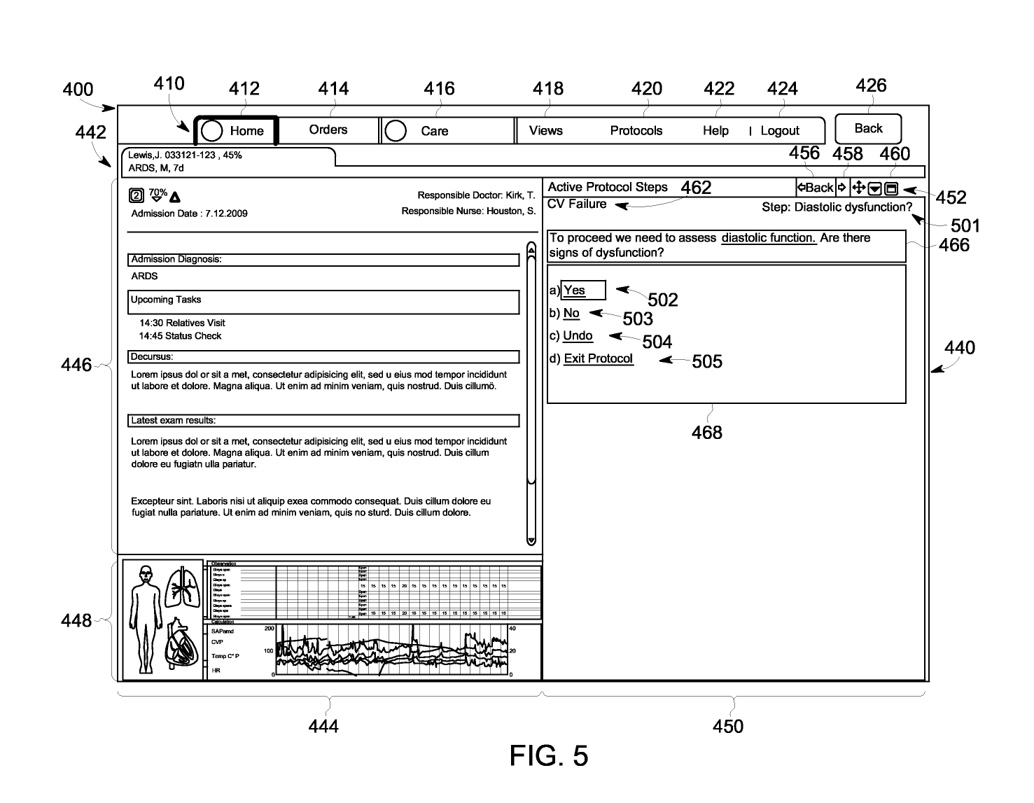

Referring to FIG. 9, block 980 generally includes determining a treatment recommendation based at least in part on the first user-generated response (refer, for example, to FIGS. 1A and 5). FIG. 5 shows the patient screen 400 after the care provider selects option 469 (in FIG. 4). The decision module 114 generates from the CV Failure protocol's rules a second rule that includes a second plurality of treatment-observation options. The user interface 112 then displays the second rule (“Diastolic dysfunction”) 501, the active care protocol (“CV Failure”) 462, detailed information relating to the second rule (“To proceed we need to assess diastolic function. Are there signs of dysfunction?”) in the detailed information dialog 466, and the second plurality of treatment-observation options 502-505. The care provider may observe the patient and, on that basis, select treatment-observation option 502 to indicate that there are signs of dysfunction, option 503 to indicate that there are no signs of dysfunction, option 504 to return to the first rule of the CV Failure protocol (the patient screen 400 shown in FIG. 4), or option 505 to exit the CV Failure protocol. Although the CV Failure protocol includes the first set of treatment-observation options 469-472 (shown in FIG. 4) and the second set of treatment-observation options 502-505, the clinical decision support system 110 may, for example, utilize a protocol having a single set of treatment-observation options. In FIG. 5, treatment-observation option 502 is highlighted to indicate that the care provider selected option 502. The user interface 112 receives the care provider's selection of option 502.

After the care provider selects option 502, the decision module 114 determines a treatment recommendation based on the care provider's selection of option 469 (shown in FIG. 4) and selection of option 502 (shown in FIG. 5). In one example, the care protocol has one or more predetermined treatment recommendations, and each treatment recommendation corresponds to a combination of selections of treatment-observation options. In the example clinical decision support system 110, for instance, the decision module 114 may correspond the care provider's selected combination of treatment-observation options (469 and 502) to the care protocol, and select the predetermined treatment recommendation in the care protocol that corresponds to that selected combination.

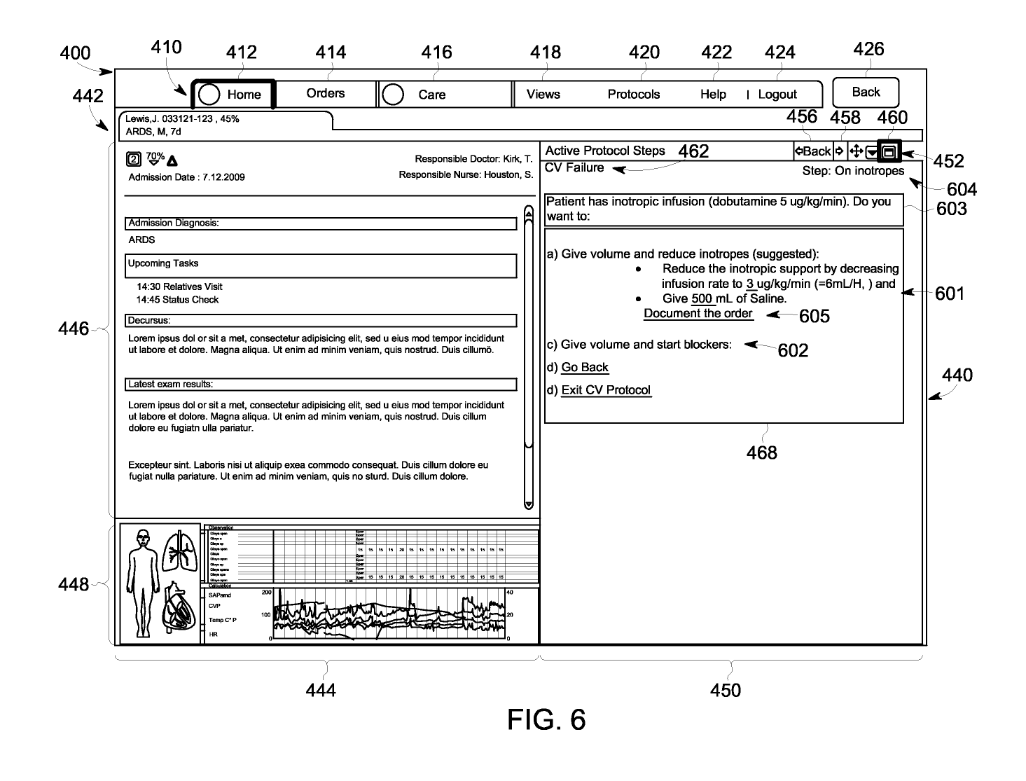

Referring to FIG. 9, block 990 generally includes displaying via the user interface the treatment recommendation to notify the care provider of the treatment recommendation for the care treatment (refer, for example, to FIGS. 1A and 6). FIG. 6 shows the patient screen 400 after the decision module 114 determines the treatment recommendation. In the patient screen 400, the user interface 112 populates the patient protocol portion 450 of the main body portion 440 with, among other things, the treatment recommendation 601, an alternate treatment recommendation 602, a diagnosis of the patient's condition 603, and the active protocol rule 604. In particular, the treatment recommendation 601 provides that the care provider should give the patient 500 ml of saline and reduce inotropic support by decreasing infusion rate to 6 mL/h The diagnosis 603 indicates that the patient has inotropic infusion. The alternate treatment recommendation 602 recommends that the care provider give the patient the stated volume and provide the patient blockers.

In an example, the user interface 112 displays treatment recommendations according to the care provider's organizational role (for example, refer to FIGS. 2 and 6). If the care provider were to enter a first role (e.g., “resident nurse”) into the role field 206, the user interface 112 may display only the treatment recommendation 601. If the care provider were to enter a second role (e.g., “non-resident nurse”) into the role field 206, the user interface 112 may display only the alternate treatment recommendation 602. If the care provider were to enter a third role (e.g., “doctor”) into the role field 206, the user interface 112 may display both the treatment recommendation 601 and the alternate treatment recommendation 602. In this way, the user interface 112 can present one or more treatment options to the care provider according to the care provider's experience level, familiarity with the patient, or the like.

Referring to FIG. 9, block 995 generally includes accepting feedback via the user interface with respect to the treatment recommendation applied to the patient (for example, refer to FIGS. 1A and 6). After the care provider implements the treatment recommendation 601, the care provider can select link 605 to document his or her order. Selecting the link 605 may, for example, send an order for recommended prescriptions to a pharmacy. Selecting the link 605 may also, or instead, update information in the general information sub-portion 446 of the patient information portion 444. Selecting the link 605 may also, or instead, enable the care provider to submit, via the user interface 112, whether and to what extent the care provider found the treatment recommendation 601 useful. The user interface 112 may accept various other forms of feedback not mentioned.

In an example, the user interface 112 displays a graphical representation of the care protocol (refer, for example, to FIGS. 6 and 7). In the patient screen 400, the care provider can select the Protocols tab 420 or the protocol display button 460 to enable the user interface 112 to display a graphical representation of the active care protocol. FIG. 7 shows the patient screen 400 displaying a flow diagram 712 of the CV Failure protocol to the care provider after the care provider selects either the Protocols tab 420 or the protocol display button 460 in the patient screen 400 shown in FIG. 6. The flow diagram 712 generally includes rule elements 732-737, treatment-option elements 738-740, and a control element 741. Each of the rule elements 732-737 corresponds to one of the CV Failure protocol's rules. For instance, the rule element 733 corresponds to the Precondition rule 464 (shown in FIG. 4), the rule element 734 corresponds to the Diastolic Dysfunction rule 501 (shown in FIG. 5), and the rule element 736 corresponds to the On Isotropes rule 604 (shown in FIG. 6). Each of the treatment-option elements 738-740 corresponds to one of the CV Failure protocol's treatment options. For instance, the treatment-option element 738 corresponds to the treatment recommendation 601 (shown in FIG. 6) and the treatment-option element 739 corresponds to the alternate treatment recommendation 602 (also shown in FIG. 6).

The flow diagram 712 also shows relationships among the rules and the treatment options. For instance, arrow 742 indicates a relationship between rule element 733 (corresponding to rule 464) and rule element 734 (corresponding to rule 501). In the example user interface 112, the care provider selected treatment-observation option 469; therefore, the rule element 734 is shaded relative to the rule element 735, and arrow 742 is solid relative to broken arrow 743. Similarly, the treatment-option element 738 is shaded relative to the treatment-option elements 739-740 to indicate that the treatment-option element 738 corresponds to the treatment recommendation 601. In this way, the care provider can visualize rules and treatment options alternative to those he or she selected. For instance, the care provider can see that were he or she to select option 470 (in FIG. 4), then the CV Failure protocol would have instructed him or her to check the patient's cardiac function, as represented by the treatment-observation element 737.

In an example, the user interface 112 can depict one or more inactive protocols. For example, refer to FIG. 7. As described above, the subsidiary protocol sub-portion 720 of the patient screen 400 includes the first flow diagram 722, the second flow diagram 724, and the third flow diagram 726. The first flow diagram 722 depicts an ARDS protocol that is in use but inactive. Likewise, the second flow diagram 724 depicts a Sedation protocol that is in use but inactive. The third flow diagram 726 depicts an Insulin protocol that is recommended and not in use. The care provider can add the Insulin protocol as an “in use” protocol by first selecting the manager link 734. The user interface 100 will then populate the patient screen 400 as shown in FIG. 8. The care provider can then select the Insulin protocol in the protocol table 810 and click the add button 818.

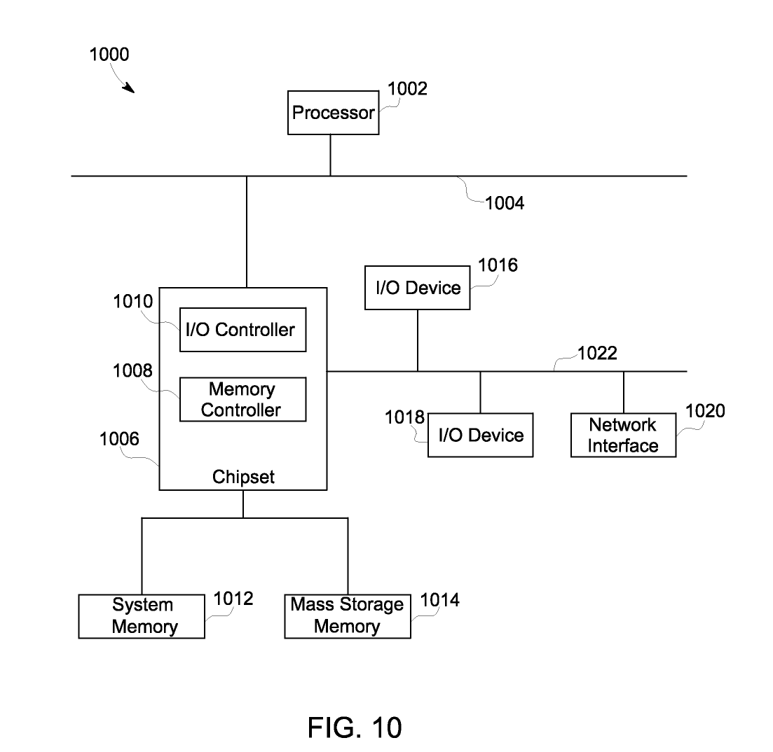

FIG. 10 is a block diagram of an example processor system 1000 that can be used to implement the apparatus and methods described herein. As shown in FIG. 10, the processor system 1000 includes a processor 1002 that is coupled to an interconnection bus 1004. The processor 1002 may be any suitable processor, processing unit, or microprocessor. Although not shown in FIG. 10, the system 1000 can be a multi-processor system and, thus, can include one or more additional processors that are identical or similar to the processor 1002 and that are communicatively coupled to the interconnection bus 1004.

The processor 1002 of FIG. 10 is coupled to a chipset 1006, which includes a memory controller 1008 and an input/output (I/O) controller 1010. As is well known, a chipset typically provides I/O and memory management functions as well as a plurality of general purpose and/or special purpose registers, timers, etc. that are accessible or used by one or more processors coupled to the chipset 1006. The memory controller 1008 performs functions that enable the processor 1002 (or processors if there are multiple processors) to access a system memory 1012 and a mass storage memory 1014.

The system memory 1012 can include any desired type of volatile and/or non-volatile memory such as, for example, static random access memory (SRAM), dynamic random access memory (DRAM), flash memory, read-only memory (ROM), etc. The mass storage memory 1014 can include any desired type of mass storage device including hard disk drives, optical drives, tape storage devices, etc.

The I/O controller 1010 performs functions that enable the processor 1002 to communicate with peripheral input/output (I/O) devices 1016 and 1018 and a network interface 1020 via an I/O bus 1022. The I/O devices 1016 and 1018 can be any desired type of I/O device such as, for example, a keyboard, a video display or monitor, a mouse, etc. The network interface 1020 may be, for example, an Ethernet device, an asynchronous transfer mode (ATM) device, an 802.11 device, a DSL modem, a cable modem, a cellular modem, etc. that enables the processor system 1000 to communicate with another processor system.

While the memory controller 1008 and the I/O controller 1010 are depicted in FIG. 10 as separate blocks within the chipset 1006, the functions performed by these blocks may be integrated within a single semiconductor circuit or may be implemented using two or more separate integrated circuits.

While the invention has been described with reference to certain embodiments, it will be understood by those skilled in the art that various changes can be made and equivalents can be substituted without departing from the scope of the invention. In addition, many modifications can be made to adapt a particular situation or material to the teachings of the invention without departing from its scope. Therefore, it is intended that the invention not be limited to the particular embodiments disclosed, but that the invention will include all embodiments falling within the scope of the appended claims.

1. A computer-implemented method to provide clinical decision support, the method comprising:
receiving a plurality of care protocols and at least one health attribute of a patient;
determining whether the at least one health attribute qualifies for a condition for which a care provider is to be notified;
providing via a user interface a patient indication, when the at least one health attribute qualifies for the condition, to notify the care provider that the patient qualifies for a care treatment;
selecting from the plurality of care protocols a first protocol that corresponds to the patient indication and that includes a first plurality of rules;
generating from the first plurality of rules a first plurality of treatment-observation options;
displaying via the user interface each of the first plurality of treatment-observation options and a suggestion regarding a recommended treatment-observation option, to enable the care provider to determine which of the first plurality of treatment-observation options applies to the patient;
receiving a first user-generated response that corresponds to at least one of the first plurality of treatment-observation options;
determining a treatment recommendation based at least in part on the first user-generated response;
displaying via the user interface the treatment recommendation to notify the care provider of the treatment recommendation for the care treatment; and
accepting feedback with respect to the treatment recommendation applied to the patient.

2. The method of claim 1, wherein determining the treatment recommendation comprises:
generating from the first plurality of rules a second plurality of treatment-observation options;
displaying via the user interface each of the second plurality of treatment-observation options, to enable the care provider to determine which of the second plurality of treatment-observation options applies to the patient;
receiving a second user-generated response that corresponds to at least one of the second plurality of treatment-observations options; and
determining the treatment recommendation based at least in part on the first and second user-generated responses.

3. The method of claim 1, further comprising displaying via the user interface a graphical representation of the first protocol.

4. The method of claim 3, wherein the graphical representation is a flow diagram that depicts each of the first plurality of rules, any relationships among each of the first plurality of rules, and the treatment recommendation.

5. The method of claim 1, further comprising displaying via the user interface a diagnosis of the condition.

6. The method of claim 1, further comprising:
determining from the at least one health attribute at least one target health attribute, and
displaying via the user interface the at least one target health attribute to notify the care provider of the at least one target health attribute that the treatment recommendation seeks to accomplish.

7. The method of claim 1, wherein providing the patient indication further comprises providing over a network the patient indication as an electronic message.

8. The method of claim 1, wherein the plurality of care protocols is generated by a care provider based on a population of patients that does not include the patient.

9. A clinical decision support system comprising:
a processor connected to a memory, the processor programmed to implement the system comprising:
a database interface to receive a plurality of care protocols and at least one health attribute of a patient;
a decision module to determine whether the at least one health attribute qualifies for a condition for which a care provider is to be notified, to select from the plurality of care protocols a first protocol that corresponds to the patient indication and that includes a first plurality of rules, to generate from the first plurality of rules a first plurality of treatment-observation options, and to determine a treatment recommendation based at least in part on a first user-generated response that corresponds to at least one of the first plurality of treatment-observation options; and
a user interface to provide the patient indication when the at least one health attribute qualifies for the condition to notify the care provider that the patient qualifies for a care treatment, to display each of the first plurality of treatment-observation options, to enable the care provider to determine which of the first plurality of treatment-observation options applies to the patient, to receive the first user-generated response, and to display the treatment recommendation to notify the care provider of the treatment recommendation for the care treatment, and to accept feedback with respect to the treatment recommendation applied to the patient.

10. The system of claim 9, wherein the decision module is to generate from the first plurality of rules a second plurality of treatment-observation options, display via the user interface each of the second plurality of treatment-observation options to enable the care provider to determine which of the second plurality of treatment-observation options applies to the patient; to receive a second user-generated response that corresponds to at least one of the second plurality of treatment-observations options, and to determine the treatment recommendation based at least in part on the first and second user-generated responses.

11. The system of claim 9, wherein the user interface is to display a graphical representation of the first protocol.

12. The system of claim 11, wherein the graphical representation is a flow diagram that depicts each of the first plurality of rules, any relationships among each of the first plurality of rules, and the treatment recommendation.

13. The system of claim 9, wherein the user interface is to display a diagnosis of the condition.

14. The system of claim 9, wherein the decision module is to determine from the at least one health attribute at least one target health attribute, and the user interface is to display the at least one target health attribute to notify the care provider of the at least one target health attribute that the treatment recommendation seeks to accomplish.

15. A tangible computer-readable storage medium comprising a set of instruction that, when executed, cause a processor to at least:
receive a plurality of care protocols and at least one health attribute of a patient;
determine whether the at least one health attribute qualifies for a condition for which a care provider is to be notified;
provide via a user interface a patient indication, when the at least one health attribute qualifies for the condition, to notify the care provider that the patient qualifies for a care treatment;
select from the plurality of care protocols a first protocol that corresponds to the patient indication and that includes a first plurality of rules;
generate from the first plurality of rules a first plurality of treatment-observation options;
display via the user interface each of the first plurality of treatment-observation options, to enable the care provider to determine which of the first plurality of treatment-observation options applies to the patient;
receive a first user-generated response that corresponds to at least one of the first plurality of treatment-observation options;
determine a treatment recommendation based at least in part on the first user-generated response;
display via the user interface the treatment recommendation to notify the care provider of the treatment recommendation for the care treatment; and
accept feedback with respect to the treatment recommendation applied to the patient.

16. The storage medium of claim 15, further comprising instructions that, when executed, cause the processor to determine the treatment recommendation by at least:
generating from the first plurality of rules a second plurality of treatment-observation options;
displaying via the user interface each of the second plurality of treatment-observation options, to enable the care provider to determine which of the second plurality of treatment-observation options applies to the patient;
receiving a second user-generated response that corresponds to at least one of the second plurality of treatment-observations options; and
determining the treatment recommendation based at least in part on the first and second user-generated responses.

17. The storage medium of claim 15, further comprising instructions that, when executed, cause the processor to at least display via the user interface a graphical representation of the first protocol.

18. The storage medium of claim 17, wherein the graphical representation is a flow diagram that depicts each of the first plurality of rules, any relationships among each of the first plurality of rules, and the treatment recommendation.

19. The storage medium of claim 15, further comprising instructions that, when executed, cause the processor to at least display via the user interface a diagnosis of the condition.

20. The storage medium of claim 15, further comprising instructions that, when executed, cause the processor to at least:
determine from the at least one health attribute at least one target health attribute, and
display via the user interface the at least one target health attribute to notify the care provider of the at least one target health attribute that the treatment recommendation seeks to accomplish.
